(19) United States
(12) Patent Application Publication
(10) Pub. No.: US 2015/0174171 A1
(43) Pub. Date: Jun. 25, 2015

(54) COMPOSITIONS AND METHODS FOR THE TREATMENT AND ALLEVIATION OF BONE AND CARTILAGE DISEASES OR INJURIES AND HAIR LOSS

(71) Applicant: Jae Woo Pak, Seoul (KR)

(72) Inventor: Jae Woo Pak, Seoul (KR)

(21) Appl. No.: 14/637,374

(22) Filed: Mar. 3, 2015

Related U.S. Application Data
(62) Division of application No. 14/142,718, filed on Dec. 27, 2013.
Application No. 14/142,718 is a division of application No. 13/413,236, filed on Mar. 6, 2012, now abandoned.
Application No. 13/413,236 is a continuation-in-part of application No. 13/166,313, filed on Jun. 22, 2011, now abandoned.
Application No. 13/166,313 is a continuation-in-part of application No. 13/035,571, filed on Feb. 25, 2011, now abandoned.

(30) Foreign Application Priority Data
Dec. 31, 2010 (KR) 10-2010-0140123

(51) Int. Cl. A61K 35/28; A61K 47/36; A61K 33/06; A61K 35/19; A61K 31/573

(52) U.S. Cl. CPC A61K 35/28; A61K 35/19; A61K 31/573; A61K 33/06; A61K 47/36

(57) ABSTRACT

The present invention provides pharmaceutical compositions, and methods of preparation and use for the treatment, prevention or alleviation of bone and cartilage diseases or injuries and hair loss. The present invention discloses a method and a pharmaceutical composition comprising mesenchymal stem cells, platelets, activating factors and scaffolding materials for the treatment, prevention, or alleviation of bone diseases, bone injuries or hair loss, and a method and a pharmaceutical composition further comprising dexamethasone for the treatment, prevention, or alleviation of cartilage diseases or injuries.

### CROSS-REFERENCE TO RELATED APPLICATIONS

The present application is a divisional of U.S. application Ser. No. 14/142,718, filed Dec. 27, 2013, which is a divisional of U.S. application Ser. No. 13/413,236, filed Mar. 6, 2012, which is a continuation-in-part of U.S. application Ser. No. 13/166,313, filed Jun. 22, 2011, which is a continuation-in-part of U.S. application Ser. No. 13/035,571, filed Feb. 25, 2011, which claims the benefit of foreign priority to South Korean Patent Application No. 10-2010-0140123, filed Dec. 31, 2010. The entire content of all of these applications is incorporated herein by reference.

### BACKGROUND OF THE INVENTION

1. Field of the Invention

The present invention relates to a pharmaceutical composition and a method for the treatment, prevention, or alleviation of cartilage diseases, cartilage injuries, bone diseases, bone injuries and hair loss comprising mesenchymal stem cells, platelets, calcium ions or other activating factors, and scaffolding materials such as hyaluronic acid, fibrin matrix, collagen, or biocomparable polymers. The present invention also relates to a pharmaceutical composition and a method further comprising dexamethasone for better improvement of the treatment, prevention, or alleviation of hyaline cartilage diseases or injuries.

2. Description of the Related Art

Osteoarthritis is a degenerative disease typically affecting adult patients, for whom the risk of onset increases with age. However, more recently, this disease has also begun to present itself in younger patients. Osteoarthritis is caused by the degradation of the joint tissue, in many cases due to the wearing down of the joint cartilage, and causes mild to severe pain. Conventionally, the damaged tissue or the whole damaged articulation is removed during the treatment of diseases caused by meniscus damage or degenerative arthritis. However, those types of surgeries include the use of surgical incisions which require long periods of recovery.

Mesenchymal stem cells (MSCs) are found in numerous human tissues including bone marrow, synovial tissue and adipose tissue. These have been shown to differentiate, in vitro and in vivo, into bones, cartilage, muscle and adipose tissue, representing a promising new therapy in regenerative medicine (Arnoczky, 1999). Because of their potent capabilities, MSCs have been used successfully in animal models to regenerate cartilage and bones (Barry, 2003; Zhang et al., 2009). In 2008, Centeno and his group reported regeneration of knee cartilage in a human by using autologous culture expanded bone marrow-derived stem cells (Centeno et al., 2008).

Adipose tissue-Derived Stem Cells (ADSCs), which is a form of mesenchymal stem cells, have been widely used in Korea over the last few years by plastic surgeons as a semi-permanent volume expander. In June 2009, the Korean Food and Drug Administration (KFDA) allowed ADSCs to be used as autologous cell transplants when obtained and processed within the same medical clinic using minimal processing.

Korean Publn. 10-2003-15160 discloses a composition comprising cell ingredients (mesenchymal stem cells, precursor cells which are derived from the mesenchymal stem cells and can be differentiated into cartilage cells or bone cells, which were differentiated into cartilage cells, or bone cells), medium and a biocompatible polymer for treating articular cartilage damage.

Korean Publn. 10-2005-64068 discloses a method for use of composition for treating cartilage comprising a) a step of preparing chondrocyte ingredients isolated and proliferated from the cartilage of a host, like human etc.; b) a step of preparing thrombin; c) a step of preparing fibrinogen matrix; d) a step of processing cartilage damage; e) a step of collecting periosteum; f) a step of stitching up the periosteum to the damage part; and g) a step injecting the composition consisting of chondrocyte ingredients, thrombin, and fibrinogen matrix to the damaged region positioned in the inside of the periosteum.

Korean Patent 10-803576 discloses a transplanting composition for plastic surgery comprising adipose stem cells, adipose cells, adipose tissue or the mixture thereof, and biodegradable stabilizers selected from the group consisting of hyaluronic acid and collagen in a physiologically suitable buffer liquid.

U.S. Pat. No. 7,803,787 discloses compositions, and methods of use thereof for treating connective tissue damage in humans and animals, for example, osteoarthritis, rheumatoid arthritis, osteochondrosis dissecans, cartilage damage, joint inflammation, joint synovitis, joint injuries, degenerative joint disease, etc., wherein, the composition comprises a therapeutically effective amount of chondroitin sulfate, N-acetyl D-glucosamine, and hyaluronan.

U.S. Pat. No. 7,078,232 discloses methods and compositions for the use of adipose tissue derived adult stem or stromal cells in combination with biocompatible, restorable and non-reabsorbable materials for the repair of articular cartilage fractures, wherein the compositions of the biocompatible liquid is selected from the group consisting of alginate, collagen, fibrin, hyaline, or plasma.

Also, U.S. Pat. Nos. 7,033,587, 6,429,013, and 6,841,150 disclose compositions and/or methods, wherein adipose tissue-derived stromal cells are differentiated into chondrocytes for the treatment cartilage repair, but not particularly related to the present invention.

Osteonecrosis is a disease where there is cellular death (necrosis) of bone components due to the interruption of the blood supply. The disease interrupts rotation or bending of articular parts, and mainly occurs in growth plate or the lower parts of an articular.

Korean Patent 10-0278905 discloses a composition for the treatment of defects in bone comprising a matrix or matrix-forming material used to fill a defect in bone, angiogenic factor at an appropriate concentration to stimulate the formation and ingrowth of blood vessels and associated cells in the matrix and the area of the defect, and an osteogenic factor associated with a delivery system and at an appropriate concentration such that upon delivery of the osteogenic factor to cells in the matrix and defect, the cells differentiate into bone cells, wherein the angiogenic factor is selected from the group consisting of bFGF, a mixture of bFGF and heparin sulfate, TGF-β, PDGF, TNFα, angiogenin, angiotropin or combinations thereof.

Korean Patent Publn. 10-2007-0121669 discloses pharmaceutical compositions for the use of inducing bone and/or cartilage formation in wound healing and tissue repair which comprises a NELL peptide or a NELL RNA. The invention discloses a method of identifying a modulator of a receptor of a NELL related peptide, comprising: contacting a receptor molecule of a NELL peptide with a test compound, contacting the NELL peptide with the receptor molecule and the test compound, detecting the extent of binding of the NELL peptide to the receptor molecule with the test compound, comparing the extent of binding of the NELL peptide to the receptor molecule with the test compound with the extent of binding of a control wherein the control is obtained by detecting the extent of binding of the NELL peptide to the receptor molecule without the test compound, and designating the test compound as a modulator of the receptor of the NELL peptide if the extent of binding of the NELL peptide to the receptor molecule with the test compound is different from the extent of binding of the control.

U.S. Pat. No. 7,807,461 relates to multipotent adult stem cells derived from human adipose tissue, particularly a method for differentiation of adult stem cells into nerve cells, fat cells, cartilage cells, osteogenic cells and insulin-releasing pancreatic beta-cells, a cellular therapeutic agent for treating osteoarthritis, osteoporosis, and diabetes, and a cellular therapeutic agent for forming breast tissue.

U.S. Pat. No. 6,391,297 discloses a method of differentiating adipose stromal cells into osteoblasts, comprising: culturing said cells in a composition which comprises a medium capable of supporting the growth of fibroblasts and differentiation inducing amounts of β-glycerophosphate and ascorbic acid and/or ascorbic-2 phosphate.

PCT Appln. WO2010/089379 relates to tissue regeneration. The invention discloses and claims a platelet-rich-plasma (PRP) and a coagulation factor as a medicament, use of PRP and a coagulation factor for the manufacture of a medicament to enrich a stem cell pool, preferably a pool of endothelial progenitor cells and/or mesenchymal stem cells of a mammal subject, use of the composition for a treatment and/or a prevention of a cardiovascular disease, cardiomyopathy, (congenital) heart disease, coronary artery disease ischemia, diastolic dysfunction, atherosclerosis, myocarditis, endocarditis and/or myocardial infarction, neurological disease, Parkinson disease, Alzheimer's disease, Huntington's disease, Tay-Sachs disease, spinal cord injury, stroke, cerebrovascular accident and amyotrophic lateral sclerosis (ALS), auto-immune disease, aplastic anemia, multiple sclerosis, rheumatoid arthritis, Sjögren's syndrome, diabetes mellitus 1, Graves' disease and/or lupus erythematosus, bone generative disease, bone atrophy, osteoporosis, bone reconstruction and bone reinforcement. The invention describes that the composition of the invention can treat and/or prevent such extensive diseases as shown above. But the invention does not disclose the ratio of the ingredients and does not show the effect either.

PCT Appln. WO2010/065854 also relates to tissue regeneration. The invention relates to an intervertebral disc as a whole.

Although there has been a lot of research and several patent applications regarding bone regeneration and cartilage regeneration, until now, there has been no successful results in humans such as those of the present invention.

The referenced shortcomings are not intended to be exhaustive, but rather are among many that tend to impair the effectiveness of previously known techniques in the prior art; however, those mentioned here are sufficient to demonstrate that the methodologies appearing in the art have not been satisfactory and that a significant need exists for the techniques described and claimed in this disclosure.

### SUMMARY OF THE INVENTION

The present invention provides a pharmaceutical composition for treatment, prevention, or alleviation of bone diseases. The present invention provides a method for treating, preventing, or alleviating bone diseases. The present invention provides a pharmaceutical composition for treatment, prevention, or alleviation of cartilage diseases. The present invention provides a method for treating, preventing, or alleviating cartilage diseases. The present invention provides a pharmaceutical composition for treatment, prevention, or alleviation of hair loss. The present invention provides a method for treating, preventing, or alleviating hair loss. The present invention provides a method for extracting adipose-derived stem cells for use in the treatment, prevention or alleviation of bone diseases, bone injuries, cartilage diseases, cartilage injuries or hair loss. Other features and associated advantages will become apparent with reference to the following detailed description of specific embodiments in connection with the accompanying drawings.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows the pictures taken by MRI before the surgical procedure and 3 months after the surgical procedure.

FIG. 2 shows the pictures taken by MRI before the surgical procedure and 3 months after the surgical procedure.

FIG. 3 shows the pictures taken by MRI before the surgical procedure and 3 months after the surgical procedure.

FIG. 4 shows the pictures taken by MRI before the surgical procedure and 3 months after the surgical procedure.

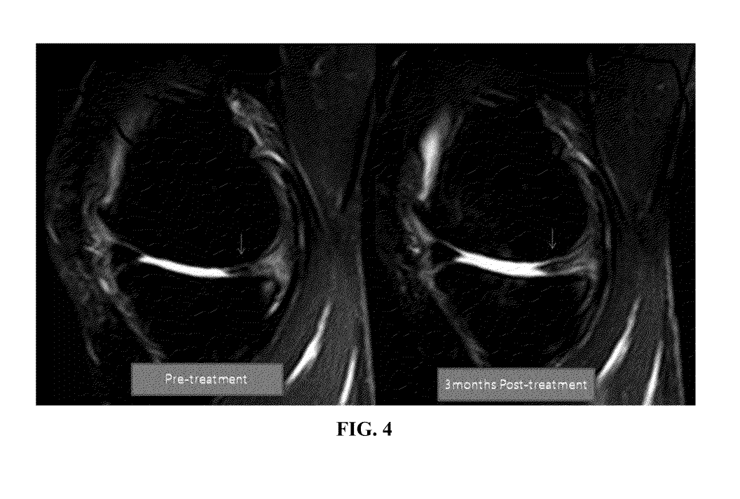

FIG. 5 shows the pictures taken before the surgical procedure and 6 weeks after the surgical procedure.

### DESCRIPTION OF ILLUSTRATIVE EMBODIMENTS

Various features and advantageous details are explained more fully with reference to the non-limiting embodiments that are illustrated in the accompanying drawings and detailed in the following description. Descriptions of well-known starting materials, processing techniques, components, and equipment are omitted so as not to unnecessarily obscure the invention in detail. It should be understood, however, that the detailed description and the specific examples, while indicating embodiments of the invention, are given by way of illustration only, and not by way of limitation. Various substitutions, modifications, additions, and/or rearrangements within the spirit and/or scope of the underlying inventive concept will become apparent to those skilled in the art from this disclosure.

While making and using of various embodiments of the present invention are discussed in detail below, it should be appreciated that the present invention provides many applicable inventive concepts, which can be embodied in a wide variety of specific contexts. The specific embodiments described herein are merely illustrative of specific ways to make and use the invention and do not delimit the scope of the present invention nor the scope of the claims appended hereto.

As used herein, bone refers to a calcified connective tissue primarily comprising a network of deposited calcium and phosphate in the foam of hydroxyapatite, collagen (predominantly type I collagen) and bone cells, such as osteoblasts and osteoclasts.

As used herein, cartilage refers to a type of connective tissue that contains chondrocytes embedded in an intercellular material (often referred to as the “cartilage matrix”) comprising fibrils of collagen (predominantly type II collagen along with other minor types, e.g., types IX and XI), various proteoglycans (e.g., chondroitinsulfate-, keratansulfate-, and dermatansulfate proteoglycans), other proteins, and water. Cartilage as used herein includes articular, hyaline, and meniscal cartilage. Articular cartilage covers the surfaces of the portions of bones in joints and allows movement in joints without direct bone-to-bone contact, and thereby prevents wearing down and damage to apposing bone surfaces. Most normal healthy articular cartilage is also described as “hyaline”, i.e., having a characteristic frosted glass appearance. Meniscal cartilage is usually found in joints, which are exposed to concussion as well as movement. Such locations of meniscal cartilage include the temporo-mandibular, sterno-clavicular, acromio-clavicular, wrist and knee joints (Gray's Anatomy, New York: Bounty Books, 1977).

Adipose tissue is an abundant source of mesenchymal stem cells (MSCs). MSCs are multipotent stem cells that are capable of differentiating into a variety of cells, including bone cells (osteoblasts), cartilage cells (chondrocytes) and fat cells (adipocytes). Although adipose tissue is one of the richest sources of MSCs that is known, MSCs can also be derived from bone marrow, placenta, lung, blood, including umbilical cord blood, and synovial tissue.

Adipose-Derived Stem Cells (ADSCs) refers to multipotent stromal cells, or stem cells that originate from adipose tissue and are capable of self-renewal, including mesenchymal stem cells. “Adipose” means any fat tissue. The adipose tissue may be brown or white adipose tissue, derived from subcutaneous, omental/visceral, mammary, gonadal, or other adipose tissue sites. Such cells may comprise a primary cell culture or an immortalized cell line. The adipose tissue may be from any organism having fat tissue.

Platelets contain growth factors and differentiating agents for promoting growth and differentiation of MSCs. Platelets contain growth factors such as transforming growth factor (TGF-β), insulin-like growth factor (IGF), fibroblast growth factor (FGF), and platelet-derived growth factor (PDGF). Platelets can be isolated and maintained in various forms, including platelet-rich plasma, platelet lysate, platelet gel, and packed platelets. Platelet-rich plasma (PRP) is an enriched platelet-containing mixture, isolated from whole blood, which is re-suspended in a small volume of plasma. While whole blood may contain about 95% red blood cells, about 5% platelets and less than 1% white blood cells, PRP may contain, for example, 95% platelets with 4% red blood cells and 1% white blood cells. PRP can be combined with activating agents such as thrombin, which activate the platelets to release their contents such as cytokinins and other growth factors. PRP also provides red blood cells necessary for formation of fibrin matrix. PRP has been used in medicine, primarily in bone grafting and dental implant applications and as part of a composition to use as a surgical adhesive. For example, Landesberg et al. (U.S. Pat. No. 6,322,785) disclose an autologous platelet gel that includes PRP for bone grafts and dental implants. The PRP is activated by collagen and is applied topically to promote wound healing. Antanavich et al. (U.S. Pat. No. 5,585,007) disclose preparation of PRP and use as a tissue sealant. Cochrum (U.S. Pat. No. 5,614,214) discloses a biopolymer that optionally includes PRP and its use to temporarily block arteries and veins. Gordinier et al. (U.S. Pat. No. 5,599,558) disclose a platelet releasate product, which includes platelets buffered to approximately pH 6.5, for use in a topical application to wounds.

To activate platelets, activating factors may be used. These activating factors aid in the occurrence of thrombosis, and include calcium, collagen, vitamin K, various tissue factors (TFs), prothrombin, thrombin and fibrinogen. For platelet injection, existing bodily collagen may itself act as an activating factor and cause platelets to release growth factors. This, for example, occurs after an injury. When there is damage to tissue, platelets are recruited by the body and activated by collagen to release growth factors.

Scaffolding materials may be added to help MSCs attach and engraft. Scaffolding materials include, but are not limited to fibrin matrix, collagens, and biocomparable polymers. Another possible scaffolding material is hyaluronic acid (also known as “Hyaluronan” or HA). It is well-known that the human body naturally contains such HA, as it is found in several parts of the body such as the soft connective tissue, the vitreous body of the eye, hyaline cartilage, synovial joint fluid, the dermis, and the epidermis. Within these parts of the body, HA acts as a lubricant between connective tissues of the skin, protects the joints by providing shock-absorption, and helps the body retain skin moisture. Over time, however, as the body ages, the amount of HA present in the body deteriorates and the body may eventually develop one of several health problems, in part due to a decreased presence of HA. This effect is particularly apparent for those who are over the age 50. Generally, the skin loses viscoelasticity, and wrinkles form ultimately as a result of this deficiency. In the present invention, hyaluronic acid is used for a scaffold. The average molecular weight may be 200,000-400,000.

Dexamethasone has been known as a differentiating agent, which promotes mesenchymal stem cells to differentiate into hyaline cartilage. Low doses of dexamethasone increase extracellular matrix production by chondrocytes, and are commonly used in vitro to differentiate MSCs into hyaline cartilage. Dexamethasone may be dexamethasone alcohol or in the form of a pharmaceutically acceptable salt or ester. Suitable salts and esters include the acetate, isonicotinoate, phenylpropionate, pivalate, t-butyl acetate, trioxaundecanoate, disodium meta-sulphobenzoate and disodium phosphate. In the present invention, dexamethasone is used as a differentiation inducer of cartilage cells.

In one aspect, the present invention provides a pharmaceutical composition for treating bone diseases and injuries comprising mesenchymal stem cells, platelets, platelet-activating factors, and scaffolding materials. By using the composition, it is possible to regenerate bone without side effects, without incisions or wounds, and hence allowing for a quicker recovery.

In another aspect, the present invention provides a pharmaceutical composition for regenerating a hipbone defect due to osteonecrosis. In still another aspect, the present invention provides a method for treating, preventing, or alleviating cartilage diseases, cartilage injuries, bone diseases, bone injuries or hair loss.

In some embodiments, the present invention provides a pharmaceutical composition for cartilage disease or injury comprising mesenchymal stem cells, platelets, platelet-activating factors, scaffolding materials and dexamethasone injected at the later time. By using the composition, it is possible to regenerate cartilage without side effects, without incisions or wounds, and hence allowing for a quicker recovery.

In some embodiments, the present invention provides a pharmaceutical composition for regenerating knee cartilage due to cartilage damage.

In some embodiments, the present invention provides a method for treating, preventing, or alleviating cartilage diseases or injuries.

Some embodiments provide a composition for the treatment, prevention, or alleviation of any of the following: bone disease, bone injury, cartilage disease, cartilage injury or hair loss comprising 1-15 parts by weight of mesenchymal stem cells, 1-10 parts by weight of platelets, 0.1-0.4 parts by weight of a 1-5% of platelet-activating factors, and 0.5-2 parts by weight of scaffolding materials, including hyaluronic acid.

Some embodiments also provide a composition for hyaline cartilage disease or damage treatment further comprising 0.05-0.2 parts by weight of dexamethasone.

According to the inventor's research, the above ingredients and the ratio have shown the most preferable effects. Also, the most effective concentration of calcium chloride used as an activating factor was 3%.

The amount of the compositions to be administered may be varied, as can the way the composition is administered and the frequency, based on, for example, whether or not the treatment or therapy is of a certain disease, the seriousness and history of the disease, the subject's age, height, weight, health state, or other conditions. Generally, heavier subjects need to be administered a larger amount.

The present invention further provides a method for the treatment of bone disease. In one embodiment, the composition comprising ADSCs, PRP, calcium chloride and hyaluronic acid is administered in a pharmaceutically effective amount to a subject in need of bone treatment and articular and meniscal cartilage treatment. In one embodiment, the composition comprising ADSCs, PRP, calcium chloride, hyaluronic acid, and later injected dexamethasone is administered in a pharmaceutically effective amount to a subject in need of (articular and meniscal) hyaline cartilage treatment. The subject may be a mammal.

At clinical trial, non-oral administration, such as injection, can be used. In case of formulation, diluents or excipients, such as filler, bulking agent, binder, wetting agent, surfactant, etc. can be used. In the formulation, sterilized water, suspension agent, emulsion, freeze-drying agent, etc. can be used. Non-aqueous solvent, suspension solvent vegetable oils, such as propylene glycol, polyethylene glycol, olive oil, or ethylolate can be used. Suppository material, such as witepsol, tween 61, stearic acid, lauric aldehyde, glycerol, or gelatin can be used.

The numerical ranges of each ingredient were determined by the treatment results of the patients treated by the present inventor's clinic.

The treatment result is shown at Table 1 after 1 g of ADSCs, 2 g of PRP, 0.2 g of CaCl₂, and 1 g of hyaluronic acid were injected to the patient of case I of the Example below, and all other methods were performed the same as the patient of case I.

TABLE 1
Functional Rating Index (12) and Visual Analog Score

| Outcome Measures | Pre-Injection | 4 week post | 12 week post |
|---|---|---|---|
| Functional Rating Index | 15 | 14 | 14 |
| VAS | 7 | 6 | 6 |

Therefore, one can see that the effect of the above ingredient ratio was not sufficient, although showed some improvement.

The treatment result is shown at Table 2 after 4 g of ADSCs, 2 g of PRP, 0.2 g of CaCl₂, and 1 g of hyaluronic acid were injected to the patient of case I of the Example below, and all other methods were performed the same as the patient of case I.

TABLE 2
Functional Rating Index (12) and Visual Analog Score

| Outcome Measures | Pre-Injection | 4 week post | 12 week post |
|---|---|---|---|
| Functional Rating Index | 15 | 13 | 10 |
| VAS | 7 | 5 | 4 |

Therefore, one can see that the effect of the above ingredient ratio was somewhat improved, even though not satisfied when compared with Table 19.

The treatment result is shown at Table 3 after 6 g of ADSCs, 0.5 g of PRP, 0.2 g of CaCl₂, and 1 g of hyaluronic acid were injected to the patient of case I of the Example below, and all other methods were performed the same as the patient of case I.

TABLE 3
Functional Rating Index (12) and Visual Analog Score

| Outcome Measures | Pre-Injection | 4 week post | 12 week post |
|---|---|---|---|
| Functional Rating Index | 15 | 14 | 13 |
| VAS | 7 | 6 | 6 |

Therefore, one can see that the effect of the above ingredient ratio was not sufficient, although showed some improvement.

The treatment result is shown at Table 4 after 6 g of ADSCs, 1 g of PRP, 0.2 g of CaCl₂, and 1 g of hyaluronic acid were injected to the patient of case I of the Example below, and all other methods were performed the same as the patient of case I.

TABLE 4
Functional Rating Index (12) and Visual Analog Score

| Outcome Measures | Pre-Injection | 4 week post | 12 week post |
|---|---|---|---|
| Functional Rating Index | 15 | 13 | 12 |
| VAS | 7 | 6 | 5 |

Therefore, one can see that the effect of the above ingredient ratio was somewhat improved, even though not satisfied when compared with Table 19.

The treatment result is shown at Table 5 after 6 g of ADSCs, 2 g of PRP, 0.05 g of CaCl₂, and 1 g of hyaluronic acid were injected to the patient of case I of the Example below, and all other methods were performed the same as the patient of case I.

TABLE 5
Functional Rating Index (12) and Visual Analog Score

| Outcome Measures | Pre-Injection | 4 week post | 12 week post |
|---|---|---|---|
| Functional Rating Index | 15 | 12 | 11 |
| VAS | 7 | 6 | 5 |

Therefore, one can see that the effect of the above ingredient ratio was not sufficient, although showed some improvement.

The treatment result is shown at Table 6 after 6 g of ADSCs, 2 g of PRP, 0.1 g of CaCl₂, and 1 g of hyaluronic acid were injected to the patient of case I of the Example below, and all other methods were performed the same as the patient of case I.

TABLE 6
Functional Rating Index (12) and Visual Analog Score

| Outcome Measures | Pre-Injection | 4 week post | 12 week post |
|---|---|---|---|
| Functional Rating Index | 15 | 12 | 9 |
| VAS | 7 | 5 | 4 |

Therefore, one can see that the effect of the above ingredient ratio was somewhat improved, even though not satisfied when compared with Table 19.

The treatment result is shown at Table 7 after 6 g of ADSCs, 2 g of PRP, 0.2 g of CaCl₂, and 0.1 g of hyaluronic acid were injected to the patient of case I, and all other methods were performed the same as the patient of case I.

TABLE 7
Functional Rating Index (12) and Visual Analog Score

| Outcome Measures | Pre-Injection | 4 week post | 12 week post |
|---|---|---|---|
| Functional Rating Index | 15 | 14 | 14 |
| VAS | 7 | 6 | 6 |

Therefore, one can see that the effect of the above ingredient ratio was not sufficient, although showed some improvement.

The treatment result is shown at Table 8 after 6 g of ADSCs, 2 g of PRP, 0.2 g of CaCl₂, and 0.5 g of hyaluronic acid were injected to the patient of case I, and all other methods were performed the same as the patient of case I.

TABLE 8
Functional Rating Index (12) and Visual Analog Score

| Outcome Measures | Pre-Injection | 4 week post | 12 week post |
|---|---|---|---|
| Functional Rating Index | 15 | 13 | 11 |
| VAS | 7 | 5 | 4 |

Therefore, one can see that the effect of the above ingredient ratio was somewhat sufficient, even though not satisfied when compared with Table 19.

The reason why the lower limits of each ingredient of the composition are determined is based on the above results. As to the upper limits of each ingredient, generally, it is known in the fields of tissue regeneration that the effects of treatment results are the better when ADSCs and PRP are used in higher amounts. However, when the two ingredients are used more than that of the upper limits, patients said it was uncomfortable. Therefore, if any patient is willing to handle discomfort, greater amounts than the upper limits can be used.

The treatment result is shown at Table 9 after 6 g of ADSCs, 2 g of PRP, 0.5 g of CaCl₂, and 1 g of hyaluronic acid were injected to the patient of case I of the Example below, and all other methods were performed the same as the patient of case I.

TABLE 9
Functional Rating Index (12) and Visual Analog Score

| Outcome Measures | Pre-Injection | 4 week post | 12 week post |
|---|---|---|---|
| Functional Rating Index | 15 | 12 | 12 |
| VAS | 7 | 6 | 5 |

Therefore, one can see that the effect of the above ingredient ratio was not sufficient, although showed some improvement.

The treatment result is shown at Table 10 after 6 g of ADSCs, 2 g of PRP, 0.4 g of CaCl₂, and 1 g of hyaluronic acid were injected to the patient of case I, and all other methods were performed the same as the patient of case I.

TABLE 10
Functional Rating Index (12) and Visual Analog Score

| Outcome Measures | Pre-Injection | 4 week post | 12 week post |
|---|---|---|---|
| Functional Rating Index | 15 | 12 | 12 |
| VAS | 7 | 6 | 5 |

Therefore, one can see that the effect of the above ingredient ratio was somewhat improved, even though not satisfied when compared with Table 19.

The treatment result is shown at Table 11 after 6 g of ADSCs, 2 g of PRP, 0.2 g of CaCl₂, and 2.5 g of hyaluronic acid were injected to the patient of case I of the Example below, and all other methods were performed the same as the patient of case I.

TABLE 11
Functional Rating Index (12) and Visual Analog Score

| Outcome Measures | Pre-Injection | 4 week post | 12 week post |
|---|---|---|---|
| Functional Rating Index | 15 | 11 | 11 |
| VAS | 7 | 6 | 5 |

Therefore, one can see that the effect of the above ingredient ratio was not sufficient, although showed some improvement.

The treatment result is shown at Table 12 after 6 g of ADSCs, 2 g of PRP, 0.2 g of CaCl₂, and 2 g of hyaluronic acid were injected to the patient of case I of the Example below, and all other methods were performed the same as the patient of case I.

TABLE 12
Functional Rating Index (12) and Visual Analog Score

| Outcome Measures | Pre-Injection | 4 week post | 12 week post |
|---|---|---|---|
| Functional Rating Index | 15 | 11 | 11 |
| VAS | 7 | 6 | 5 |

Therefore, one can see that the effect of the above ingredients' ratio were somewhat improved, even though not satisfied when compared with Table 19.

The treatment result is shown at Table 13 after 6 g of ADSCs, 2 g of PRP, 0.2 g of CaCl₂, 1 g of hyaluronic acid, and 0.03 g of dexamethasone (dexamethasone injected at the week number 2) were injected to the patient of case III of the Example below, and all other methods were performed the same as the patient of case I.

TABLE 13
Functional Rating Index (12) and Visual Analog Score

| Outcome Measures | Pre-Injection | 4 week post | 12 week post |
|---|---|---|---|
| Functional Rating Index | 36 | 32 | 31 |
| VAS | 7 | 6 | 6 |

Therefore, one can see that the effect of the above ingredient ratio was not sufficient, although showed some improvement.

The treatment result is shown at Table 14 after 6 g of ADSCs, 2 g of PRP, 0.2 g of CaCl₂, 1 g of hyaluronic acid, and 0.05 g of dexamethasone (dexamethasone injected at the week number 2) were injected to the patient of case III, and all other methods were performed the same as the patient of case I.

TABLE 14
Functional Rating Index (12) and Visual Analog Score

| Outcome Measures | Pre-Injection | 4 week post | 12 week post |
|---|---|---|---|
| Functional Rating Index | 36 | 28 | 21 |
| VAS | 7 | 4 | 4 |

Therefore, one can see that the effect of the above ingredients' ratio were somewhat improved, even though not satisfied when compared with Table 21.

The treatment result is shown at Table 15 after 6 g of ADSCs, 2 g of PRP, 0.2 g of CaCl₂, 1 g of hyaluronic acid, and 0.3 g of dexamethasone (dexamethasone injected at the week number 2) were injected to the patient of case III of the Example below, and all other methods were performed the same as the patient of case III.

TABLE 15
Functional Rating Index (12) and Visual Analog Score

| Outcome Measures | Pre-Injection | 4 week post | 12 week post |
|---|---|---|---|
| Functional Rating Index | 36 | 35 | 33 |
| VAS | 7 | 6 | 5 |

Therefore, one can see that the effect of the above ingredient ratio was not sufficient, although showed some improvement.

The treatment result is shown at Table 16 after 6 g of ADSCs, 2 g of PRP, 0.2 g of CaCl₂, 1 g of hyaluronic acid, and 0.2 g of dexamethasone (dexamethasone injected at the week number 2) were injected to the patient of case III of the Example below, and all other methods were performed the same as the patient of case III.

TABLE 16
Functional Rating Index (12) and Visual Analog Score

| Outcome Measures | Pre-Injection | 4 week post | 12 week post |
|---|---|---|---|
| Functional Rating Index | 36 | 35 | 33 |
| VAS | 7 | 6 | 5 |

Therefore, one can see that the effect of the above ingredients' ratio were somewhat improved, even though not satisfied when compared with Table 21.

Even though in the examples below only human's bone regeneration, cartilage regeneration and hair regrowth are described, it is expected that the composition may have the same or similar effect to connective tissues in animals.

### EXAMPLES

The invention is now further described by reference to the following examples, which are intended to illustrate, not to limit the scope of the invention.

This series of case reports represents successful clinical results of regenerating bones in osteonecrosis patients, articular and meniscus cartilage in osteoarthritis and hair in hair loss patients, using percutaneously implanted, autologous MSCs along with PRP, hyaluronic acid, calcium chloride (for bone, cartilage, and hair regeneration) and further comprising dexamethasone (for hyaline cartilage generation).

### Example 1

Clinical case-reports I and II show that a combination of percutaneously injected autologous ADSCs, PRP, hyaluronic acid, and calcium chloride can regenerate bones in human beings caused by osteonecrosis.

Stem cells were obtained from adipose tissue of the abdominal origin by digesting the lipoaspirate tissue with collagenase enzyme. These stem cells, along with hyaluronic acid, PRP and calcium chloride were injected at hip bones. Before and after the surgical procedure, the hip bone areas were analyzed by MRI image, physical therapy, and pain score data.

Patients' MRI images showed a big difference in the size of the hip bones. Also, the results of physical therapy, pain score and functional rating index were all improved. Therefore, it is considered that surgical operation or incision could be replaced with the injection of the composition of the present invention for the regeneration of bones.

### Case I

The patient is a 29-year-old Korean female with more than a one year history of right hip pain. Approximately 1 year prior to the visit, the patient started having the hip pain without any history of trauma. She was seen by a physician and was diagnosed with osteoarthritis of hip, after an MRI. After taking NSAIDS for few weeks, the hip pain improved until about 1 month prior to my clinic visit. Again, the patient started having the hip pain radiating to the anterior region of the right knee. The pain was worse when standing up, walking and exercising. However, the pain alleviated with rest. The pain was not much relieved with NSAIDS, this time.

Repeated MRI showed osteonecrosis of femoral head, stage 4. Since there has been no effective non-surgical treatment of the disease, the patient elected to receive a stem cell treatment of the present invention. At the time of initial evaluation, the patient reported moderately severe pain (VAS score 7) on rest, increased pain when standing and walking (VAS score 9).

### Liposuction Procedure:

For one week prior to the liposuction, the patient was restricted from taking corticosteroids, aspirin, NSAIDs, and oriental herb medications.

For the liposuction procedure, the patient was brought to an operating room and was placed in a supine position. Then, the patient was sedated with Propofol 2 mg IV push and 20 mg/hour rate of continuous infusion.

After cleaning the abdominal area with povidone-iodine and placing sterile drapes, an incision of approximately 0.5 cm was made about 5 cm below the umbilicus. Then, using Tumnescent solution (500 cc NS+40 cc of 2% Lidocaine+20 cc of 0.5% Marcaine+0.5 cc of Epinephrine 1:1000), the lower abdomen area was anesthetized. Next, using a 3.0 Hartman cannula, a total of 160 cc of lipoaspirates were extracted and separated by gravity. The resulting 100 cc of adipose tissue was then centrifuged at 3500 rpm for 5 minutes. The end result was approximately 40 cc of packed adipose tissue, fibrous tissue, RBC's and a small number of nucleated cells.

The digestive enzyme, collagenase, was then mixed with the centrifuged lipoaspirates at a ratio of 1:1 and digested for 30 min at 37° Celsius while rotating.

After the digestion, the lipoaspirates were centrifuged at 100 g for 3 minutes to separate the lipoaspirate and the enzyme. The left-over enzyme was then removed.

Using 500 cc D5LR, the lipoaspirates were washed three times to remove the collagenase. After each washing, the lipoaspirates were centrifuged at 100 g. After the last centrifuge, approximately 6 g of ADSCs were obtained. As would be understood to a person of ordinary skill upon reading this procedure, at this point, the ADSCs are contained in a stromal vascular fraction (SVF). This ADSC-containing SVF is referred to as ADSCs in the illustrative examples for simplicity.

### PRP Preparation:

While preparing the ADSCs, 30 cc of autologous blood was drawn with 2.5 cc of Anticoagulant Citrate Dextrose Solution (ACD) formula. This was centrifuged at 200 g for 5 min. The resultant supernatant was drawn and spun at 1000 g for 5 minutes. The supernatant was drawn and discarded. Among the resulting buffy coat, 2 g was taken and mixed with 6 g of ADSC.

To this mixture, hyaluronic acid 1 g was added as a scaffold. This PRP was again mixed with 0.2 g of CaCl₂ for activation of platelets at a ratio of 10:2 (PRP 10:2 CaCl₂).

### Injection of the Composition:

In order to inject the mixture of stem cells and PRP, the patient was, first, placed in a lateral position with her left side down. After cleaning with povidone-iodine and draping with sterile drapes, 2% lidocaine was used to anesthetize the hip at the femoral head region. Using a 22-gauge 3½ inch needle, 17 cc mixture of ADSCs, PRP, hyaluronic acid and CaCl₂ were injected into the femoral head under the ultrasound guidance.

The patient was, then, instructed to remain still with her leg elevated for 30 minutes to allow for cell attachment. As the patient was discharged to home, the patient was instructed to maintain activity as tolerated.

The patient returned for 4 additional PRP (4 cc) injections with calcium chloride (0.8 cc) every week over 1 month period.

### Results:

After the 4th week following the composition injection, the patient's pain improved more than 50%. By the 12th week, the patient's pain improved more than 70%.

TABLE 17
Functional Rating Index (12) and Visual Analog Score

| Outcome Measures | Pre-Injection | 4 week post | 12 week post |
|---|---|---|---|
| Functional Rating Index | 15 | 12 | 8 |
| VAS | 7 | 4 | 2 |

TABLE 18
Physical Therapy-Range of Motion

| | Flexion | Flexion | Abduction | Abduction | Adduction | Adduction |
|---|---|---|---|---|---|---|
| Pt Session | (deg) | VAS | (deg) | VAS | (deg) | VAS |
| Pre-injection evaluation | 91 | 5 | 20 | 6 | 10 | 7 |
| 4 week post injection | 110 | 3 | 35 | 3 | 15 | 3 |
| 12 week post injection | 125 | 2 | 40 | 2 | 20 | 2 |

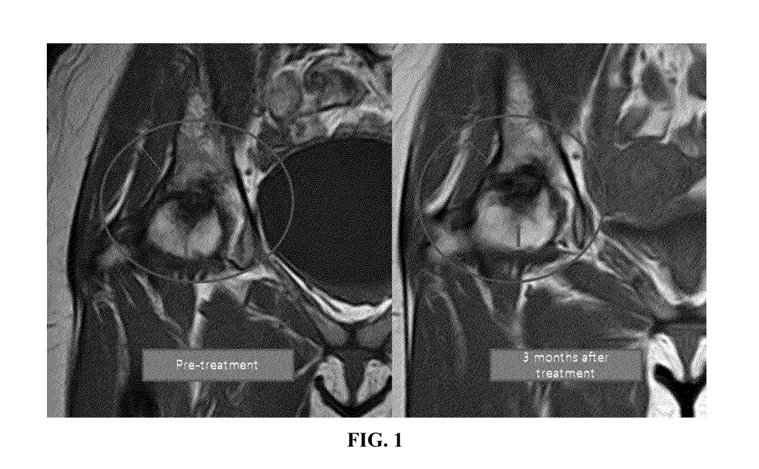

Repeated MRI taken at the 12th week showed a significant filling of bone defects on the superior acetabulum and probable bone matrix formation at the subcortical region of femoral head. (FIG. 1)

FIG. 1 is the MRI image of right hip, T1 coronal section. Lesions on superior acetabulum and subcortical head of femur have decreased in size. Bone regeneration at superior acetabulum (↓) and consolidation of bone matrix at the subcortical region of head of femur are evident ( [symbol] , ↑).

### Case II

The patient is a 47-year-old Korean male who has been working as a diver until 3 years prior to my clinic visit. Approximately, 3 year ago he started having right hip pain and was diagnosed with osteonecrosis of right hip. The patient's pain has progressed over three years and the patient was offered a total hip replacement (THR). Being reluctant with the surgical procedure, the patient elected to go with the stem cell treatment of the present invention. Before the procedure, MRI was taken and the patient was diagnosed with osteonecrosis of femoral head, stage 4.

Liposuction, PRP preparation, and the composition injection were operated as case I. The patient returned for 4 additional PRP (4 cc) injections with calcium chloride (0.8 cc) every week over 1 month period.

### Results:

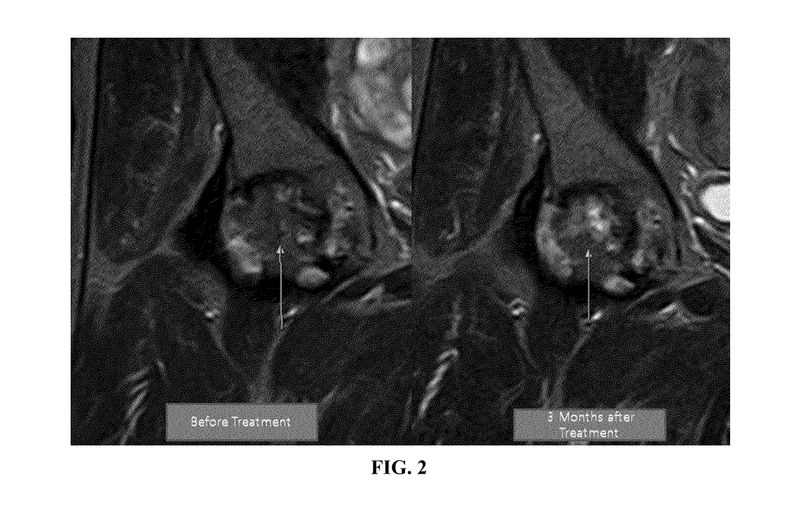

After the 4th week following the composition injection, the patient's pain improved more than 30%. However, by the 12th week, the patient's pain minimally alleviated further. Interestingly, repeat MRI taken at the 12th week showed a significant filling of bone defects with a possibility of bone matrix formation at the site of necrosis in the femoral head (FIG. 2).

TABLE 19
Functional Rating Index and Visual Analog Score

| Outcome Measures | Pre-Injection | 4 week post | 12 week post |
|---|---|---|---|
| Functional Rating Index | 16 | 12 | 12 |
| VAS | 8 | 5 | 5 |

TABLE 20
Physical Therapy-Range of Motion

| | Flexion | Flexion | Abduction | Abduction | Adduction | Adduction |
|---|---|---|---|---|---|---|
| PT Session | (deg) | VAS | (deg) | VAS | (deg) | VAS |
| Pre-injection evaluation | 90 | 8 | 15 | 8 | 10 | 8 |
| 4 week post injection | 100 | 5 | 20 | 5 | 10 | 5 |
| 12 week post injection | 105 | 5 | 20 | 5 | 15 | 5 |

FIG. 2 is the MRI image of right hip, T2 coronal view. Possible bone matrix regeneration is evident in the 3 months post-treatment.

### Example 2

Clinical case-reports III and IV showing a combination of percutaneously injected autologous ADSCs, PRP, hyaluronic acid, calcium chloride and dexamethasone (dexamethasone injected at the week number 2) could be able to regenerate cartilage in human osteoarthritis.

### Case III

The patient is a 70-year-old Korean female with more than a 5 year history of right knee pain. The patient has been a long time farm worker with active use of bilateral knee joints.

With the diagnosis of osteoarthritis of the right knee, she had received multiple injections of steroids and hyaluronic acid over the last few years. However, she did not notice any improvement of the pain. The patient was seen by an orthopedic surgeon and was offered a Total Knee Replacement (TKR). She was reluctant to go through the TKR due to possible side effects. Since then, the patient had been receiving physical therapy without much improvement.

At the time of initial evaluation, the patient reported moderately severe pain (VAS score 7) on rest, increased pain when walking. The patient also complained of mild knee swelling.

On physical examination, there was mild joint edema, decreased range of motion and tenderness with flexion. Apley's and McMurray's tests were negative, and there was no ligament laxity.

A pre-treatment 1.5 T MRI demonstrated a decreased size and deformed contour on medial meniscus of the left knee due to maceration.

Liposuction and PRP preparation were performed as case I.

In order to inject the composition comprising 6 g of ADSCs, 2 g of PRP, 1 g of hyaluronic acid, and 0.2 g of calcium chloride, the patient was, first, placed in a supine position with her right knee bent at 90°. After cleaning with povidone-iodine and draping with sterile drapes, the knee was anesthetized with 2% lidocaine at the medial and lateral sides of the inferior patella. Using a 22-gauge 1 inch needle, total 8.5 cc of the composition was injected into each side of medial and lateral knee.

The patient was, then, instructed to remain still for 30 minutes to allow for cell attachment. As the patient was discharged from the clinic, the patient was instructed to maintain activity as tolerated.

The patient returned for four additional 4 cc of PRP and 0.2 g of calcium chloride injections over 4 weeks. 0.8 cc of dexamethasone was injected at week 2 of PRP and calcium chloride injection.

### Results:

After the 7th week following the ADSC injection, the patient's pain was alleviated by more than 80% and the flexion of the knee improved as well. By the 12th week, the patient's pain improved more than 90% and the range of motion also further improved.

TABLE 21
Functional Rating Index and Visual Analog Score

| Outcome Measures | Pre-Injection | 4 week post | 12 week post |
|---|---|---|---|
| Functional Rating Index | 36 | 16 | 13 |
| VAS | 7 | 2 | 1 |

TABLE 22
Physical Therapy-Range of Motion

| | VAS | Flexion | Flexion | Extension | Extension |
|---|---|---|---|---|---|
| PT Session | Score | (deg) | VAS | (deg) | VAS |
| Pre-injection | 7 | 110 | 7 | +3 | 1/10 |
| evaluation | | | | | |
| 7 week post | 2 | 130 | 3 | +5 | 0/10 |
| injection | | | | | |
| 12 week post | 1 | 130 | 2 | +5 | 0/10 |
| injection | | | | | |

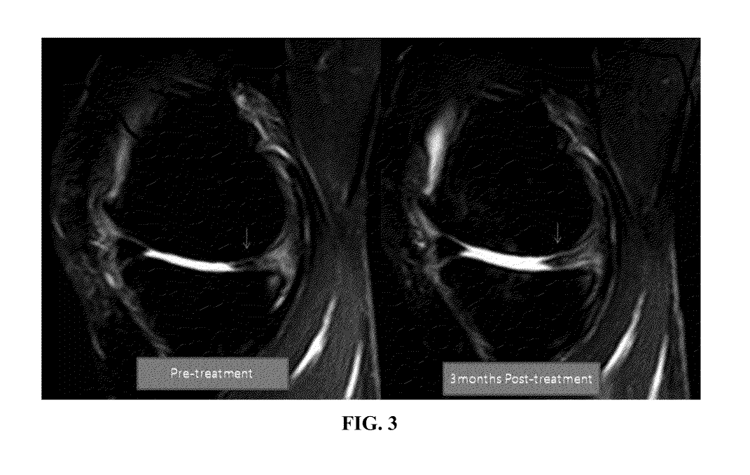

Post treatment MRI taken at the 12th week showed a significant increase in the thickness of meniscus cartilage on the medial side of the right knee (FIG. 3).

FIG. 3 is MRI Sagittal T2 view. Pre- and post treatment MRI shows increased height of medial meniscus cartilage and articular cartilage (arrow).

### Case IV

The patient is a 79-year-old Korean female with more than a 7 year history of bilateral knee pain. The left knee is much more painful than the right. The patient has been a housewife throughout her life. With the diagnosis osteoarthritis of bilateral knees, she received multiple injections of steroids and hyaluronic acid on both knees over the last few years. However, the patient noticed no improvement of pain. The patient was seen by an orthopedic surgeon and was offered a Total Knee Replacement (TKR). This patient was also reluctant to go through the TKR due to possible side effects. Since then, the patient was receiving physical therapy without much improvement.

At the time of initial evaluation, the patient reported severe pain on the left knee (VAS score 8) on rest, increased pain when walking.

On physical examination, there was deformity of the knee, mild joint swelling, decreased range of motion and tenderness with flexion. Apley's and McMurray's tests were negative, and there was no ligamentous laxity.

A pre-treatment 1.5 T MRI demonstrated a decreased size and deformed contour on medial meniscus of the left knee due to maceration. Liposuction and PRP preparation and injection of the composition of case IV were performed as in case III.

### Results:

At the 4th week after ADSC injection, the patient's pain improved over 50% and the flexion of the knee improved as well. By the 12th week, the pain improved over 90% and the patient was able to flex the knee further.

TABLE 23
Functional Rating Index and Visual Analog Score

| Outcome Measures | Pre-Injection | 4 week post | 12 week post |
|---|---|---|---|
| Functional Rating Index | 36 | 20 | 13 |
| VAS | 8 | 4 | 1 |

TABLE 24
Physical Therapy-Range of Motion

| | | Flexion | Flexion | Extension | Extension |
|---|---|---|---|---|---|
| PT Session | Vas Score | (deg) | VAS | (deg) | VAS |
| Pre-injection | 8 | 110 | 7 | +3 | 1/10 |
| evaluation | | | | | |
| 4 week post | 4 | 120 | 5 | +4 | 0/10 |
| injection | | | | | |
| 12 week post | 1 | 130 | 2 | +5 | 0/10 |
| injection | | | | | |

### Example 3

Clinical case-report V shows that a combination of percutaneously injected autologous ADSCs, PRP, hyaluronic acid and calcium chloride could be able to regrow hair in patients suffering from alopecia.

### Case V

The patient is a 53-year-old Korean male with androgenic alopecia, categorized as Type III Vertex on the Norwood-Hamilton Scale of Male Pattern Baldness. This patient has not undergone any other treatments for this condition.

Liposuction was performed as in case I.

### PRP Preparation:

While preparing the ADSCs, 60 cc of autologous blood was drawn with 9.0 cc of Anticoagulant Citrate Dextrose Solution (ACD) formula. This was centrifuged at 200 g for 10 min. The resultant supernatant was drawn and spun at 1000 g for 5 minutes. The supernatant was drawn and discarded. Among the resulting buffy coat, 8 g was taken and mixed with 8 g of ADSC.

This mixture was aliquoted into 2 equal portions. To the second aliquot of this mixture, 1 g of hyaluronic acid was added as a scaffold. To both aliquots, the PRP was again mixed with 0.8 g of 3% CaCl₂ for activation of platelets at a ratio of 100:2 (PRP 10:2 CaCl2).

### Injection of the Composition:

In order to inject the mixture of stem cells and PRP, the patient was, first, placed in a supine position with his backside down. After cleaning with povodine-iodine and draping with sterile drapes, 2% lidocaine was used to anesthetize the scalp area at the vertex. Using a 23-gauge 1 inch needle, 8.8 cc of the mixture of ADSCs, PRP and CaCl₂ were injected into the designated areas of the scalp on the left side of the vertex and 9.8 cc of the mixture of ADSCs, PRP, hyaluronic acid and CaCl₂ were injected into the designated areas of the scalp on the right side of the vertex.

The patient was, then, instructed to remain still with his head elevated for 30 minutes to allow for cell attachment. As the patient was discharged to home, the patient was instructed to maintain activity as tolerated.

The patient returned to clinic in one week (i.e. 7 days later) for additional PRP (8 cc from 60 cc of the whole blood drawn) injections with calcium chloride (1.6 cc). The patient again returned to clinic two weeks later (i.e., 21 days from the initial ADSC injection) for the 3rd dose of PRP (8 cc from 60 cc of whole blood drawn) injections with calcium chloride (1.6 cc).

### Results:

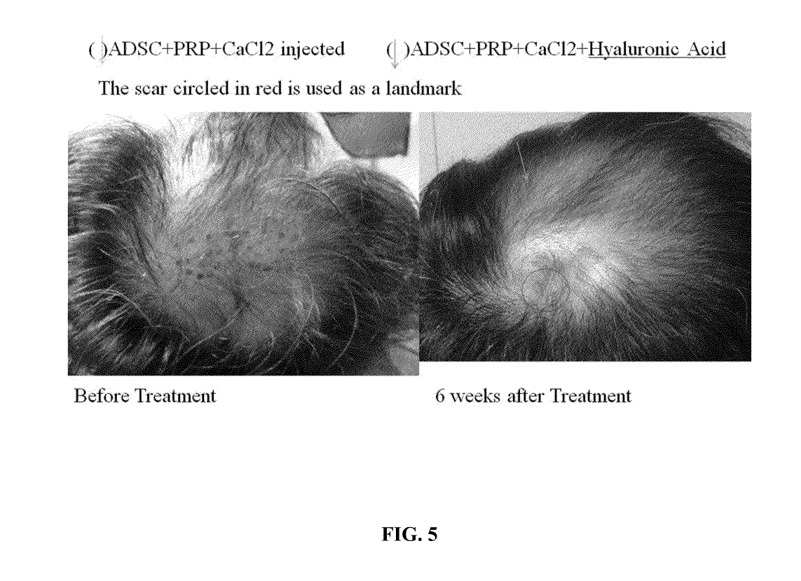

After 6 weeks from the initial injection, the patient showed a significant increase in hair thickness in both the areas to the left and right of the cranial vertex. In the area to the right of the vertex, the area treated with the mixture that included hyaluronic acid, the patient showed a significant increase in the number of hair follicles in addition to the thickening of the existing hair. (FIG. 5)

Modifications and other embodiments of the invention will become apparent to one skilled in the art to which this invention pertains having the benefit of the teachings presented in the foregoing descriptions. It is to be understood that the invention is not limited to the specific embodiments disclosed and that modifications and other embodiments are intended to be included within the scope of the appended claims.

### REFERENCES

The following references, to the extent that they provide exemplary procedural or other details supplementary to those set forth herein, are specifically incorporated herein by reference:
U.S. Pat. No. 5,585,007
U.S. Pat. No. 5,599,558
U.S. Pat. No. 5,614,214
U.S. Pat. No. 6,322,785
U.S. Pat. No. 6,391,297
U.S. Pat. No. 6,429,013,
U.S. Pat. No. 6,841,150
U.S. Pat. No. 7,033,587
U.S. Pat. No. 7,078,232
U.S. Pat. No. 7,803,787
U.S. Pat. No. 7,807,461
Arnoczky, Clin. Orthop. Relat. Res., 367:S244-253, 1999.
Barry, Novartis Found. Symp., 249:86-96, 2003.
Centeno et al., Pain Physician, 11:3:343-353, 2008.
Gray's Anatomy, Bounty Books, NY, 1977.
Korean Patent 10-0278905
Korean Patent 10-803576
Korean Publn. 10-2003-15160
Korean Publn. 10-2005-64068
Korean Publn. 10-2007-0121669
PCT Appln. WO2010/065854
PCT Appln. WO2010/089379
Zhang et al., Chin. J. Traumata, 12:92-97, 2009.

What is claimed is:

1. A method for using a pharmaceutical composition to treat or alleviate cartilage disease or cartilage injury in humans comprising:
administering an effective amount of the pharmaceutical composition, wherein the pharmaceutical composition comprises 1 to 12 parts by weight of adipose-derived stem cells.

2. The method of claim 1, wherein the pharmaceutical composition further comprises 1 to 2 parts by weight of platelet-rich plasma.

3. The method of claim 2, wherein the pharmaceutical composition further comprises platelet-activating factors.

4. The method of claim 2, wherein the pharmaceutical composition further comprises a solution containing 1 to 5% calcium ions.

5. The method of claim 4, wherein the pharmaceutical composition further comprises hyaluronic acid.

6. The method of claim 5, wherein the hyaluronic acid comprises 0.5 to 2 parts by weight of the pharmaceutical composition.

7. The method of claim 1, wherein the pharmaceutical composition further comprises 0.05 to 0.2 parts by weight of dexamethasone.
